(19) United States
(12) Patent Application Publication
(10) Pub. No.: US 2006/0074312 A1
(43) Pub. Date: Apr. 6, 2006

(54) Medical diagnostic ultrasound signal extraction

(76) Inventors: Bogdan Georgescu, Princeton, NJ (US); Xiang Zhou, Exton, PA (US); Dorin Comaniciu, Princeton Jct., NJ (US); Sriram Krishnan, Exton, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/186,717

(22) Filed: Jul. 21, 2005

Related U.S. Application Data
(60) Provisional application No. 60/616,279, filed on Oct. 6, 2004.

(51) Int. Cl. A61B 8/00

(52) U.S. Cl. 600/437

(57) ABSTRACT

Ultrasound signal information is detected from a sequence of images. A robust automated delineation of the border of the fan or ultrasound signal information in echocardiographic or other ultrasound image sequence is provided. The processor implemented delineation uses a single image or a sequence of images to better identify ultrasound signal data. Variation through a sequence generally identifies the signal area. Projecting the filtered variation information to two likely directions identifies approximate edge locations along the sides of the border. Robust regression fits lines to the edges to find accurate border locations. The bottom of the border is identified with a histogram of the variation information as a function of radius from an intersection of the fit lines.

### RELATED APPLICATIONS

The present patent document claims the benefit of the filing date under 35 U.S.C. §19(e) of Provisional U.S. Patent Application Ser. No. 60/616,279, filed Oct. 6, 2004, which is hereby incorporated by reference.

### BACKGROUND

This present embodiments generally relate to extraction of imaging information. Images generated by x-ray systems, such as mammograms, are analyzed by a computer to assist in diagnosis. The images typically include four views taken on a same day. In addition to image information representing x-ray signals used to scan a patient, the images also include textual or other information related to the patient or the scan. For computer assisted diagnosis, the textual or other information my result in inaccurate analysis of the x-ray signal data. Various filters are applied to extract the x-ray signal data.

In ultrasound, the imaging system composites textual or other information with the ultrasound signal data. The resulting image or sequence of images is displayed to the user for diagnosis. For computer assisted diagnosis, the ultrasound signal data is analyzed for wall motion tracking, detection, global motion compensation or other analysis.

### BRIEF SUMMARY

By way of introduction, the preferred embodiments described below include methods, systems or computer readable media for detecting ultrasound signal information from a sequence of images. A robust automated delineation of the border of the fan or ultrasound signal information in echocardiographic or other ultrasound image sequence is provided. Other medical information may be identified. The processor implemented delineation uses a single image or a sequence of images to better identify ultrasound signal data.

In a first aspect, a method is provided for detecting ultrasound image information from images. A first image including ultrasound information in a first portion and other information in a second portion is obtained. The first image is processed with a processor to identify the first portion.

In a second aspect, a computer readable storage media has stored therein data representing instructions executable by a programmed processor for detecting ultrasound signal information from a sequence of images. The images include the ultrasound signal information and other information (e.g., textual, background or textual and background information). Data for the image is without a data indication distinguishing the ultrasound signal information from the other information. The storage media comprising instructions for: identifying a border for the ultrasound signal information in the images, and extracting the ultrasound signal information within the border.

In a third aspect, a system is provided for detecting ultrasound signal information from a sequence of images. A memory is operable to store a sequence of images. Each image includes the ultrasound signal information and other information in different first and second portions. A processor is operable to extract the ultrasound signal information from within a border.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system for detecting ultrasound signal information from an image;

FIG. 2 is a flow chart diagram of one embodiment of a method for detecting ultrasound signal information from an image;

FIG. 3 is a graphical representation of an ultrasound image in one embodiment;

FIG. 4 is a graphical representation of data variation through a sequence of images in one embodiment;

FIG. 5 is a graphical representation of locations identified by directional filtering in one embodiment;

FIG. 6 is a graphical representation of one embodiment of a histogram; and

FIG. 7 is a graphical representation of one embodiment of a fan region of the image of FIG. 3.

### DETAILED DESCRIPTION OF THE DRAWINGS AND PRESENTLY PREFERRED EMBODIMENTS

For computer assisted analysis or diagnosis of ultrasound signal information, the data in an image associated with imaging or signals received in response to an acoustic scan is identified. Data associated with background, such as a black background, and text is removed or not used. The computer assisted diagnosis algorithm operates on the ultrasound signal information without confusion, errors or reduced efficiency by also operating on non-signal information.

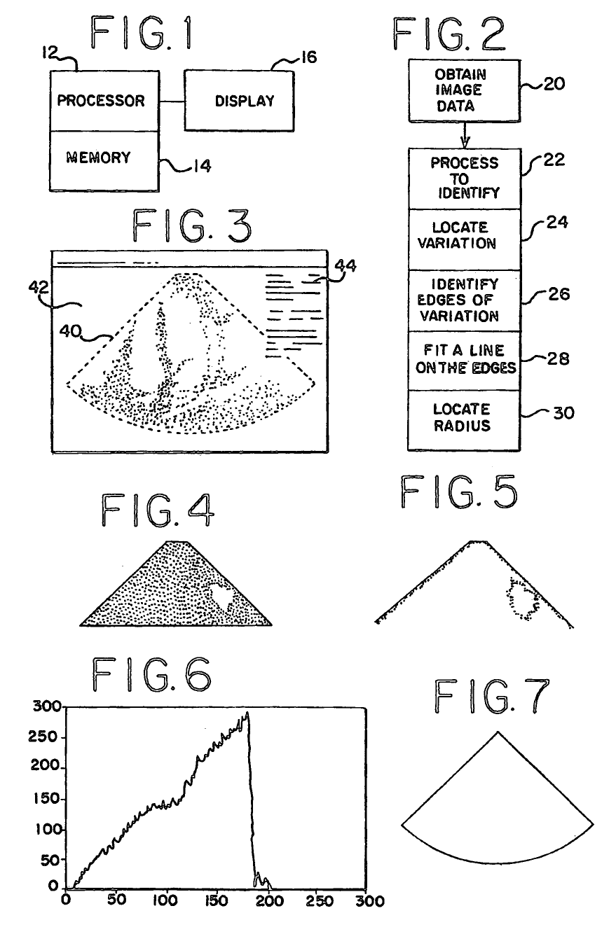

FIG. 1 shows a system 10 for detecting ultrasound signal information from a sequence of images. The system 10 includes a processor 12, a memory 14 and a display 16. Additional, different or fewer components may be provided. In one embodiment, the system 10 is a medical diagnostic imaging system, such as an ultrasound imaging system. In other embodiments, the system 10 is a computer, workstation or server. For example, a local or remote workstation receives images for computer assisted diagnosis. The system 10 identifies portions of the image associated with ultrasound signal information for subsequent automatic diagnosis. The system 10 may alternatively identify portions of a medical image associated with magnetic resonance, computed tomography, nuclear, positron emission, x-ray, mammography or angiography.

The processor 12 is one or more general processors, digital signal processors, application specific integrated circuits, field programmable gate arrays, servers, networks, digital circuits, analog circuits, combinations thereof, or other now known or later developed device for processing medical image data. The processor 12 implements a software program, such as code generated manually or programmed or a trained classification or model system. The software identifies and extracts ultrasound signal information from one or more images also having other information. Alternatively, hardware or firmware implements the identification.

The processor 12 is also operable to apply an image analysis algorithm to the extracted ultrasound signal information and not applying the image analysis algorithm to other information from outside the border. For example, the processor 12 is a classifier implementing a graphical model (e.g., Bayesian network, factor graphs, or hidden Markov models), a boosting base model, a decision tree, a neural network, combinations thereof or other now known or later developed algorithm or training classifier for computer assisted diagnosis. The classifier is configured or trained for computer assisted diagnosis and/or detecting ultrasound signal information. Any now known or later developed classification schemes may be used, such as cluster analysis, data association, density modeling, probability based model, a graphical model, a boosting base model, a decision tree, a neural network or combinations thereof. In other embodiments, the processor applies the image analysis algorithm based on a manually programmed algorithm. Alternatively, the processor 12 does not perform computer assisted diagnosis, but extracts the signal information for subsequent processing by another system or processor.

The processor 12 is operable to extract the ultrasound signal information from within a border. Ultrasound signal information is displayed in a fan, such as associated with sector or Vector® scans of a patient. The fan area generally includes two diverging, straight lines joined at a point or by a short line or curve at the top. A larger curve joins the lines at the lower edge. Alternatively, the ultrasound signal information is displayed in a circular area (e.g., radial scan) or a rectangular area (e.g., linear scan). Other shapes may be used. The processor 12 identifies the border to determine the location of the ultrasound signal information.

Filtering, thresholds, image processing, masking or other techniques may be used to extract the ultrasound signal. The extraction is automated, such as being performed without user input during the processing and/or without user indication of location. The techniques are applied to a single image or a sequence of images.

The memory 14 is a computer readable storage media. Computer readable storage media include various types of volatile and non-volatile storage media, including but not limited to random access memory, read-only memory, programmable read-only memory, electrically programmable read-only memory, electrically erasable read-only memory, flash memory, magnetic tape or disk, optical media and the like. The memory 14 stores the ultrasound image data for or during processing by the processor 12. The ultrasound data is input to the processor 12 or the memory 14.

The image data are RGB, gray scale, YUV, intensity, detected or other now known or later developed data values for imaging on the display 16. The image data may be in a Cartesian coordinate, polar coordinate or other format. The image data may not distinguish one portion of an image from another portion other than having different values for different pixel locations. The image data represents different types of information, such as signal information and other information (e.g., textual and/or background). Ultrasound signal information represents echoes from a scanned region. The different types of information are provided in different portions of the image. The different portions may overlap, such as textual information extending into the portion displaying ultrasound signal information, or may not overlap, such as the background being provided only where the ultrasound signal information is not.

The image data is for a single image or a plurality of images. For example, the ultrasound image data is a sequence of B-mode images representing a myocardium at different times with an associated background and textual overlay. The sequences are in a clip, such as video, stored in a CINE loop, DIACOM images or other format.

In one embodiment, the memory 14 is a computer readable storage media having stored therein instructions executable by the programmed processor 12. The automatic or semiautomatic operations discussed herein are implemented, at least in part, by the instructions. The instructions cause the processor 12 to implement any, all or some of the functions or acts described herein. The functions, acts or tasks are independent of the particular type of instructions set, storage media, processor or processing strategy and may be performed by software, hardware, integrated circuits, film-ware, micro-code and the like, operating alone or in combination. Likewise, processing strategies may include multiprocessing, multitasking, parallel processing and the like.

In one embodiment, the instructions are stored on a removable media drive for reading by a medical diagnostic imaging system or a workstation networked with imaging systems. An imaging system or work station uploads the instructions. In another embodiment, the instructions are stored in a remote location for transfer through a computer network or over telephone communications to the imaging system or workstation. In yet other embodiments, the instructions are stored within the imaging system on a hard drive, random access memory, cache memory, buffer, removable media or other device.

The instructions are for detecting ultrasound signal information from a sequence of images. The images include the ultrasound signal information and other information. The image data is a specific data indication distinguishing the ultrasound signal information from the other information. There is not data indicating any given spatial location is associated with a particular type of data. Instead, the image data is formatted to indicate a value or values at particular spatial locations.

The instructions are for identifying a border for the ultrasound signal information in the images. By identifying the border, the ultrasound signal information representing echoes from a scanned region is identified. The ultrasound signal information within the border is extracted for subsequent application of an image analysis algorithm without data from other information.

FIG. 2 shows a method for detecting ultrasound image information from an image or a sequence of images. Additional, different, or fewer acts than shown may be provided, such as processing the identify ultrasound signal information without determining a border in acts 24-30. The acts may be performed in a different order than shown, such as locating the radius in act 30 prior to identifying edges in act 26.

In act 20, at least one image is obtained. A sequence of images, such as a video of images, is obtained in one embodiment. For example, the sequence of images represents a heart of a patient over one or more heart cycles. The image is obtained from storage. The storage is part of a medical diagnostic ultrasound imaging system, a workstation, a tape or disk recording or a centralized medical record data base. The image is a previously displayed and recorded image from an imaging system. Alternatively, the image is obtained by substantially real-time transfer from or within an imaging system. The image is obtained by a processor within the imaging system or by a processor remote from the imaging system used to acoustically scan the patient.

Ultrasound information is in a first portion of each image, and other information is in a second portion of each image. The first and second portions overlap or are separate. FIG. 3 shows one embodiment of one ultrasound image. The image includes ultrasound information region 40 representing the patient. The ultrasound information region 40 is fan or Vector® shaped as shown, but may have other shapes. The ultrasound information section 40 includes data representing ultrasound signals, such as acoustic echoes. The image also includes a background section 42. The background section 42 is uniform, such as a uniform black or other color, or may include texture or other display background. The text section 44 includes graphics or textual information overlaid on the background section 42 and/or the ultrasound information section 40. The text section indicates trademark information, patient information, imaging system setting information, quantities or graphs derived from the ultrasound information or other text or graphics information.

Through a sequence of images, the border of the ultrasound information section 40, the background section 42 and the text section 44 typically stay the same, but may vary. The data representing the ultrasound information in the ultrasound information section 40 more likely varies or changes in a different way than the other sections.

In act 22, the image or images are processed with a processor, performed automatically, and/or performed pursuant to instructions in a computer readable media. The processing identifies the ultrasound information section 40 and/or the ultrasound information or data of the ultrasound information section 40. For example, the ultrasound information section 40 is automatically detected to identify the ultrasound information representing an ultrasonically scanned region.

The processing to identify the ultrasound information or section 40 uses a single image or a sequence of images. Any now known or later developed classifiers, models, filters, image processing techniques or other algorithms may be used. Acts 24-30 represent one approach using a sequence of images.

In act 24, spatial positions associated with intensity variation are located as a function of time in the sequence of images. Ultrasound signal information may vary more than background or text information from image to image in a sequence. Pixels associated with ultrasound signal information tend to vary through a sequence. For example, the scanned tissue may move (e.g., echocardiography), the transducer may move, speckle or other noise variation may exist or other signal related properties may change. Textual and/or background information vary less or are the same throughout the sequence.

FIG. 4 shows intensity variation associated with a sequence of images including the image shown in FIG. 3. The difference between sequential or other images in a sequence of images is calculated for each spatial location. A single difference is calculated or multiple differences associated with different pairs or other groupings of images are calculated. An average, maximum, minimum, median, standard deviation or other characteristic of the intensity variations is selected to provide the intensity variation value for each spatial location. As shown in FIG. 4, the textual and background information may stay the same, resulting in a zero or substantially zero intensity variation through the sequence. A threshold may be applied to map all values below the threshold to zero and/or above the threshold to a high value, such as black.

The processing of act 24 is masked in one embodiment. For example and as shown in FIG. 4, the inter-image intensity variation is calculated for each spatial location in an upper two thirds of the image. Other larger or smaller, continuous or discontinuous, and/or from different angles (e.g., side instead of top) masks may be used. Alternatively, no masking is performed, and the intensity variation is calculated for the entire image.

In act 26, the edges of the ultrasound information section 40 are identified. Points are identified along at least one edge of the ultrasound information section 40 as represented by the intensity variations shown in FIG. 4. For example, the points along the side edges are identified. As shown in FIGS. 3 and 4, the side edges extend at about 45 degree angles from vertical or horizontal. Locations or points of intensity variation associated with a transition in intensity variation along first and second angles associated with possible first and second edges of the border are determined or detected. The side edges, such as the diverging sides of a sector scan image, are within a range of angles. For example, the angles are about plus or minus 30-60 degrees where 0 degrees is horizontal for most ultrasound images. +/−45 degrees is used in one embodiment. Different angles may be used, such as generating locations for a same edge by filtering along two or more angles (e.g., 35, 45 and 55 degrees). The results may be averaged or used as independent data points. In general, a filter is applied to project data along angles likely to be about perpendicular to the possible edges. In one embodiment, a step filter (i.e., space domain profile) is applied, but other filters or algorithms may be used. FIG. 5 shows the points identified along the edges using a step filter at +/−45 degrees. By identifying a transition from variation to no variation along the possible angles, the side or other edges are more likely identified.

Since the locations may vary or not form a continuous line or curve, lines or curves are fit along the located edges or transitions in intensity variation as a function of the locations. For example, two lines along the side edges are fit based on the points identified and shown in FIG. 5. Different lines are fit for the locations associated with the different step filtering angles.

The line fitting uses any now known or later developed approach, such as linear or non-linear regression (e.g., robust regression). For one embodiment of regression, the Total Least Squares estimate is used and represented as:

$$TLS \to {arg min}_{θ} {\sum }_{i} {( \frac{{χ}_{i}^{T} θ}{‖ θ ‖} )}^{2} \qquad ( 1 )$$

where θ are the line parameters and χᵢ are measurements (homogeneous points). The Total Lease Squares provides an orthogonal regression, is unbiased and may result in a lower mean-squared error as compared to Ordinary Least Squares. Other regression, such as Ordinary Least Squares, may be used. The calculation is made robust to minimize the effects of points inside or outside of the desired border. To provide robust regression, an estimation process is included. For example, a biweight M-estimator:

$$M \text{-} estim \to {arg min}_{θ} {\sum }_{i} ρ ( {u}_{i} ) , {u}_{i} = \frac{{χ}_{i}^{T} θ}{σ ‖ θ ‖} \qquad ( 2 )$$

is used, where ρ is the robust loss function (biweight M-estimator) and σ is the error scale. The minimized error is operated on by the biweight loss function. After one or more iterations, the solution is provided by the weighted total least squares function. An initial estimate for the line location and the error scale is found by projecting the candidate points on several directions, such as +/−30, 45 and/or 60, and finding the mode and standard deviation of the point distribution (i.e., projection pursuit). In alternative embodiments, other estimators, regression or line fitting functions are used.

The bottom edge is detected for a sector scan by locating a radius from an intersection of the first and second fit lines (e.g., sides) corresponding to a curved bottom edge of the border. The greatest radius associated with a sufficient intensity variation is identified and used to define the curved bottom edge. For example, a histogram of number of pixels with sufficient intensity variation as a function of radial distance from the intersection is populated. The radius where the histogram has a decreasing value is selected as the radius defining a bottom edge of the ultrasound signal information. Other techniques using the same or different processes may be provided for sector or other scan formats.

Once the border, region, area, volume and/or spatial locations associated with ultrasound signal are identified, image analysis algorithms may be applied to the ultrasound signals without or with less interference from non-ultrasound data in the images. For example, a cardiac quantification algorithm (e.g., ejection fraction, motion analysis, segmentation or tissue boundary detection) is applied to the data within the border through the sequence of images. The same border is used throughout the sequence, but the border may vary for one or more images in the sequence. As another example, algorithms for identifying tissue borders, movement, texture, size, shape and/or other parameters used for diagnosis or computer assisted diagnosis are applied to the ultrasound data.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for detecting ultrasound image information from images, the method comprising:
obtaining a first image including ultrasound information in a first portion and other information in a second portion;
processing the first image with a processor; and
identifying the first portion with the processor based on the processing.

2. The method of claim 1 wherein obtaining the first image comprises obtaining a previously displayed image from an imaging system, the processor being part of a workstation separate from the imaging system.

3. The method of claim 1 wherein obtaining comprises obtaining a video of images including the first image and additional images.

4. The method of claim 1 wherein processing and identifying comprise automatic detecting of the first portion, the ultrasound information being data representing an ultrasonically scanned region.

5. The method of claim 1 wherein obtaining comprises obtaining a sequence of images including the first image, and wherein processing comprises locating spatial positions associated with intensity variation as a function of time.

6. The method of claim 5 wherein processing comprises calculating an inter-image intensity variation for spatial locations throughout the sequence.

7. The method of claim 6 wherein the processing is performed on an upper two thirds of the images.

8. The method of claim 5 wherein processing comprises detecting locations associated with a transition along first and second angles associated with possible first and second borders of the first portion.

9. The method of claim 8 wherein the first and second angles are about plus or minus 30-60 degrees where 0 degrees is horizontal relative to the first image.

10. The method of claim 1 wherein processing comprises:
identifying points along at least one edge of the first portion; and
fitting a line along the at least one edge.

11. The method of claim 10 wherein fitting the line comprises applying a robust regression.

12. The method of claim 1 wherein identifying comprises identifying at least two straight edges, and wherein processing comprises locating a radius of the first portion from an intersection of the two straight lines, a bottom of the first portion being defined by a radial curve at the radius.

13. The method of claim 12 wherein locating the radius comprises populating a histogram as a function of radi from the intersection and identifying the radius where the histogram has a decreasing value.

14. The method of claim 1 further comprising:
applying an image analysis algorithm to the first portion through a sequence of images.

15. In a computer readable storage media having stored therein data representing instructions executable by a programmed processor for detecting ultrasound signal information from a sequence of images, the images including the ultrasound signal information and other information comprising textual, background or textual and background information, data for the image being without a data indication distinguishing the ultrasound signal information from the other information, the storage media comprising instructions for:
identifying a border for the ultrasound signal information in the images; and
extracting the ultrasound signal information within the border.

16. The instructions of claim 15 wherein the ultrasound signal information represents echoes from a scanned region; and
further comprising:
applying an image analysis algorithm to the extracted ultrasound signal information and not applying the image analysis algorithm to the other information.

17. The instructions of claim 15 wherein identifying the border comprises:
locating spatial positions associated with intensity variation as a function of time;
detecting locations associated with a transition in intensity variation along first and second angles associated with possible first and second edges of the border;
fitting first and second lines along the first and second edges as a function of the locations; and
locating a radius from an intersection of the first and second lines corresponding to a curved bottom edge of the border.

18. The instructions of claim 17 wherein:
locating spatial positions comprises calculating an inter-image intensity variation for each spatial location in an upper two thirds of the images throughout the sequence;
detecting locations comprises detecting along the first and second angles are about plus or minus 30-60 degrees where 0 degrees is horizontal, the first and second angles being perpendicular to the possible first and second edges, respectively;
fitting comprises applying a robust regression; and
locating the radius comprises populating a histogram as a function of radi from the intersection and identifying the radius where the histogram has a decreasing value.

19. A system for detecting ultrasound signal information from a sequence of images, the system comprising:
a memory operable to store a sequence of images, each image including the ultrasound signal information and other information in different first and second portions, respectively;
a processor operable to extract the ultrasound signal information from within a border.

20. The system of claim 19 wherein the ultrasound signal information represents echoes from a scanned region; and
wherein the processor is operable to apply an image analysis algorithm to the extracted ultrasound signal information and not applying the image analysis algorithm to other information from outside the border.

21. The system of claim 19 wherein the processor is operable to:
locate spatial positions associated with intensity variation as a function of time in the sequence of images;
detect locations associated with a transition in intensity variation along first and second angles associated with possible first and second edges of the border;
fitting first and second lines along the first and second edges as a function of the locations; and
locating a radius from an intersection of the first and second lines corresponding to a curved bottom edge of the border.

22. A method for detecting ultrasound image information from images, the method comprising:
obtaining a first medical image including ultrasound, magnetic resonance, computed tomography, nuclear, positron emission, or angiography information in a first portion and other information in a second portion;
processing the first image with a processor; and
identifying the first portion with the processor based on the processing.

23. The method of claim 22 wherein the first medical image includes magnetic resonance information in the first portion.

24. The method of claim 22 wherein the first medical image includes computed tomography information in the first portion.

25. The method of claim 22 wherein the first medical image includes nuclear information in the first portion.

26. The method of claim 22 wherein the first medical image includes positron emission information in the first portion.

27. The method of claim 22 wherein the first medical image includes angiography information in the first portion.
